(19) United States
(12) Patent Application Publication
(10) Pub. No.: US 2026/0297707 A1
(43) Pub. Date: Oct. 1, 2026

(54) HIGH-STRENGTH AND LOW-MODULUS ANTIBACTERIAL ALLOY, PREPARATION METHOD THEREFOR, AND USE THEREOF

(71) Applicant: Dongfang Jiayue(Jiangsu) Medical Technology Company Ltd., Changzhou (CN)

(72) Inventors: LU WANG, Changzhou (CN); TAO ZHANG, Changzhou (CN); JING RUAN, Changzhou (CN); MEIJUN JIANG, Changzhou (CN)

(21) Appl. No.: 19/309,830

(22) Filed: Aug. 26, 2025

(30) Foreign Application Priority Data
Mar. 25, 2025 (CN) 202510357619.6

(51) Int. Cl. C22C 14/00; A61K 6/84; A61P 31/04; B05D 1/18; C22C 1/10; C22F 1/18

(52) U.S. Cl. CPC C22C 14/00; A61K 6/84; A61P 31/04; B05D 1/18; C22C 1/1036; C22F 1/183; B05D 2202/35

(57) ABSTRACT

Disclosed are a high-strength and low-modulus antibacterial alloy, a preparation method therefor, and use thereof. The high-strength and low-modulus antibacterial alloy has a molecular formula of TiₐZr_{b}Nb_{c}Cu_{d}AgₑO_{f}, where subscripts a, b, c, d, e, and f represent atomic percentages of corresponding alloy elements, with 50≤a≤70, 20≤b≤30, 5≤c≤7, 2≤d≤4, 1≤e≤5, 2≤f≤4, and a+b+c+d+e+f=100; and a remainder is an unavoidable impurity. Through component optimization and process innovation, a microstructure of the alloy is effectively controlled, thereby significantly improving mechanical properties and antibacterial properties of the alloy, solving a bottleneck problem of difficulties in achieving both mechanical properties and biological functions in traditional alloys, and providing a new-generation alloy solution for implantable medical apparatus and instruments.

### CROSS-REFERENCE TO RELATED APPLICATIONS

This application claims priority to Chinese Patent Application No. 202510357619.6, filed on Mar. 25, 2025, which is hereby incorporated by reference in its entirety.

### TECHNICAL FIELD

The present invention relates to the technical field of preparation of biomedical materials, in particular to a high-strength and low-modulus antibacterial alloy, a preparation method therefor, and use thereof.

### BACKGROUND

In the field of biomedicine, implantable medical apparatus and instruments have been widely used for treatment of diseases and functional restoration of the body. Traditional biomedical materials such as stainless steel and titanium alloys, although having high strength, have too high modulus, which are not matched with mechanical properties of natural bones of the human body and easily cause a stress shielding effect, leading to issues such as loose bone tissues and loose implants around implantation sites. In addition, most implants do not have antibacterial properties, and implant-related infections are serious clinical issues, which increase the pain and medical costs of patients and may even lead to surgical failures. In the prior art, although some high-strength and low-modulus antibacterial biomedical materials have been reported, further improvements are still needed in terms of material components, preparation processes, and performance optimization. Therefore, development of a biomedical material with superior performance and a simpler preparation process has important theoretical and practical significance.

### SUMMARY

To solve the above problems, the present invention provides a high-strength and low-modulus antibacterial alloy. The alloy has a molecular formula of TiaZrbNbcCudAgeOf, where subscripts a, b, c, d, e, and f represent atomic percentages of corresponding alloy elements, with 50≤a≤70, 20≤b≤60, 5≤c≤7, 2≤d≤4, 1≤e≤5, 2≤f≤4, and a+b+c+d+e+f=100; and a remainder is an unavoidable impurity.

Preferably, a surface of the high-strength and low-modulus antibacterial alloy is coated with a zinc-strontium three-dimensional network coating.

The present invention further provides a method for preparing the high-strength and low-modulus antibacterial alloy, which includes the following steps:
batching: weighing Ti, Zr, Nb, Ag, Cu, and TiO2 according to preset target components of the alloy, and uniformly mixing the same to obtain raw materials for melting, where purities of various elements by mass percentage are not lower than 99.0%;
preparation of an alloy ingot using a vacuum arc: placing the raw materials for melting into a vacuum arc device, performing vacuumizing and arc melting in an argon protective atmosphere, and taking out and obtaining the alloy ingot;
preparation of an alloy bar by solidification: placing the above alloy ingot into a rapid solidification device, performing vacuumizing and melting in an argon protective atmosphere, and after complete melting, performing spray casting into a copper mold for rapid cooling and solidification within 2-4 min to obtain the alloy bar, where the rapid cooling is conducive to forming a fine grain structure and improving hardness and wear resistance of the alloy;
hot working: placing the above alloy bar into a heating device, performing vacuumizing, heating up to 950-1,050° C. at 10-20° C./min under rotation in an argon protective atmosphere to ensure uniform heating of the alloy bar, performing heat preservation at such a temperature for 30-60 min, and then taking out the alloy bar for forging and rolling, where a deformation amount of the alloy bar is controlled at 10-30% during the process, an appropriate heating temperature may ensure that the alloy has good plasticity during the hot working to avoid grain growth and a decrease in alloy strength caused by overheating, an appropriate heat preservation time may ensure uniform distribution of the alloy components while avoiding grain growth caused by a too long time, and an appropriate deformation amount may refine grains to improve strength and toughness of the alloy; and
heat treatment: subjecting the hot worked alloy bar to solid solution treatment and aging treatment.

Preferably, during the preparation of the alloy ingot using the vacuum arc, a current is set at 50-120 A, a melting temperature is 1,500-2,500° C., a melting frequency is 4-5 times, and a melting time is 2-3 min each time. An appropriate current and a higher melting temperature are conducive to full melting of the alloy components to form the uniform alloy ingot. Several times of the melting within a short time may reduce the risk of oxidation of the alloy at a high temperature.

Preferably, the solid solution treatment includes the following steps: cleaning a surface of the above alloy bar to ensure that the surface is free of an impurity and an oxide layer; placing the cleaned alloy bar into a solid solution treatment device, and heating up to 850-950° C. at a heating rate of 30-40° C. per hour in an argon protective atmosphere, where the heating rate is not too high to avoid thermal stress caused by a rapid temperature change; performing heat preservation at such a temperature for 2.5-3.5 h, where a heat preservation time is not too short to ensure that solute atoms inside the alloy are fully dissolved into a matrix to form a uniform single-phase solid solution, and the heat preservation time is not too long to avoid grain growth; and after the heat preservation is completed, naturally cooling the alloy bar.

Preferably, the aging treatment includes the following steps: taking the solid solution treated alloy bar out of the solid solution treatment device, and performing surface cleaning; placing the cleaned alloy bar into an aging treatment device, heating up to 500-600° C. at a heating rate of 10-20° C. per hour in an argon protective atmosphere, and performing heat preservation at such a temperature for 3.5-4.5 h, where a heat preservation time is not too short to ensure that a precipitate phase is formed inside the alloy to further improve material strength and hardness; and after the heat preservation is completed, naturally cooling the alloy bar.

The present invention additionally provides a method for preparing the high-strength and low-modulus antibacterial alloy, which includes the following steps:
batching: weighing Ti, Zr, Nb, Ag, Cu, and TiO2 according to preset target components of the alloy, and uniformly mixing the same to obtain raw materials for melting, where purities of various elements by mass percentage are not lower than 99.0%;
preparation of an alloy ingot using a vacuum arc: placing the above raw materials for melting into a vacuum arc device, performing vacuumizing and arc melting in an argon protective atmosphere, and taking out and obtaining the alloy ingot;
preparation of an alloy bar by solidification: placing the above alloy ingot into a rapid solidification device, performing vacuumizing and melting in an argon protective atmosphere, and after complete melting, performing spray casting into a copper mold for rapid cooling and solidification within 2-4 min to obtain the alloy bar;
hot working: placing the above alloy bar into a heating device, performing vacuumizing, heating up to 950-1,050° C. at 10-20° C./min under rotation in an argon protective atmosphere to ensure uniform heating of the alloy bar, performing heat preservation at such a temperature for 30-60 min, and then taking out the alloy bar for forging and rolling, where a deformation amount of the alloy bar is controlled at 10-30% during the process;
heat treatment: subjecting the hot worked alloy bar to solid solution treatment and aging treatment; and
coating with a zinc-strontium three-dimensional network coating: uniformly mixing, in parts by mass, 25-35 parts of a 5-30 wt % calcium silicate solution, 5-9 parts of a 0.5-5.5 wt % zinc nitrate solution, and 5-11 parts of a 0.5-5.5 wt % strontium nitrate solution to form a uniform sol solution, adding 45-65 parts of ethanol to adjust viscosity and uniformity of the sol solution, immersing the high-strength and low-modulus antibacterial alloy in the sol solution for 3-5 min, then slowly lifting the antibacterial alloy to make the sol solution uniformly coated on a surface of the high-strength and low-modulus antibacterial alloy, performing natural air drying, then placing the antibacterial alloy in a heating device for heating to 600-800° C., and performing heat preservation for 1-3 h. Zinc ions in the zinc nitrate solution and strontium ions in the strontium nitrate solution undergo an ion exchange reaction with silicon-oxygen bonds in the calcium silicate solution to form stable chemical bonds, and then undergo high-temperature curing to form a stable three-dimensional network structure. Such structure, on the one hand, can increase a contact area between the coating and the alloy surface to improve a binding force between the coating and the alloy surface and reduce a risk of shedding of the coating, and on the other hand, can achieve more uniform distribution of the zinc ions and the strontium ions, thereby significantly improving hardness, wear resistance, and antibacterial properties of the alloy surface. By utilizing a synergistic effect of different sterilization mechanisms of zinc and strontium elements in the coating and copper and silver elements in the alloy to jointly improve the antibacterial properties of the alloy, it is ensured that the alloy can efficiently and rapidly kill various common pathogenic bacteria. The zinc ions can disrupt a biofilm to enhance permeability of the copper ions and the silver ions. The copper ions and the silver ions can interfere with activity of metabolic enzymes and generate reactive oxygen species, and the strontium ions can interfere with bacterial calcium signaling pathways, thus forming a multi-target attack on bacteria. The coating and the matrix synergistically regulate ion release rates to ensure a long-lasting antibacterial effect.

### Beneficial Effects:

According to the technical solution, through component optimization and process innovation, further optimization of mechanical properties and antibacterial efficacy of a medical alloy is achieved. By adding the titanium dioxide as a controllable oxygen source to generate nano-oxides with other components in the alloy under high temperature conditions, these oxides may serve as second-phase particles for refining alloy grains to enable the alloy to achieve a yield strength of more than 1,000 MPa, a fracture strength of more than 1,500 MPa, and a decreased elastic modulus of less than 55 GPa, thereby significantly alleviating a stress shielding effect. Oxygen vacancies generated by thermal decomposition of the titanium dioxide promote sustained release of the silver ions and the copper ions, and combined with photocatalytic activity of the titanium dioxide, an inhibition rate against Staphylococcus aureus reaches more than 96%. By adopting a dual regulation mechanism of internal oxygen supply (thermal decomposition of TiO₂) and external oxygen blocking (vacuum and argon protection), the content of oxygen in the alloy is stably controlled within a target range, thereby avoiding grain boundary embrittlement and meanwhile ensuring uniform distribution of an oxide strengthening phase. By precisely regulating key process parameters such as a heating temperature and a heating time during the vacuum arc melting, the rapid solidification, the hot working, and the heat treatment, a microstructure of the alloy is effectively controlled, thereby significantly improving mechanical properties of the alloy. Through the above multi-dimensional innovations, the high-strength and low-modulus highly efficient antibacterial alloy is obtained, thereby solving a bottleneck problem of difficulties in achieving both mechanical properties and biological functions in traditional alloys, and providing a new-generation alloy solution for implantable medical apparatus and instruments.

### BRIEF DESCRIPTION OF THE DRAWINGS

Drawings described herein are used to provide a further understanding of the present application and constitute a part of the present application, and schematic embodiments and descriptions thereof are used to explain the present application and are not intended to constitute undue limitations to the present application. In the drawings:

FIG. 1 is an X-ray diffraction (XRD) pattern of alloys prepared in Examples 1-3 of the present invention.

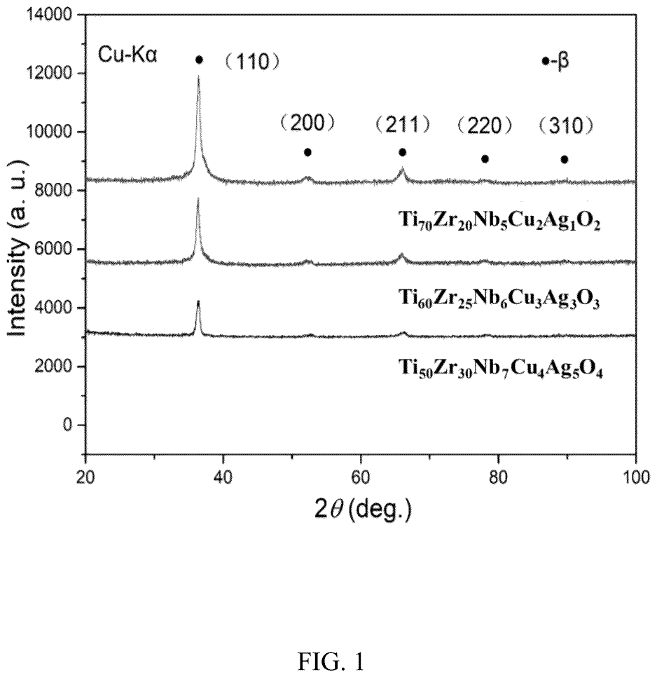

### DESCRIPTION OF EMBODIMENTS

In order to make the technical solutions of the present invention clearer and more explicit to those skilled in the art, the present invention is further described in detail below in conjunction with the embodiments and drawings, but the embodiments of the present invention are not limited thereto.

### Example 1

### Preparation of a Ti70Zr20Nb5Cu2Ag1O2 Alloy

Batching: 3336.42 g of a titanium elementary substance, 1842.83 g of a zirconium elementary substance, 469.24 g of a niobium elementary substance, 128.38 g of a copper elementary substance, 108.96 g of a silver elementary substance, and 80.68 g of titanium dioxide were weighed and uniformly mixed to obtain raw materials for melting, where purities of various elements by mass percentage were not lower than 99.0%.

Preparation of an alloy ingot using a vacuum arc furnace: The raw materials for melting were placed into the vacuum arc furnace, vacuumizing was performed to 5×10-3 Pa, argon was introduced, arc melting was performed in an argon protective atmosphere, and the alloy ingot was taken out and obtained, where a current was set at 50 A, a melting temperature was 1,500° C., a melting frequency was 4 times, and a melting time was 2 min each time.

Preparation of an alloy bar by solidification: The alloy ingot was placed into a rapid solidification device, vacuumizing was performed to 5×10-3 Pa, argon was introduced, the alloy ingot was heated to 1,500° C. in an argon protective atmosphere, and after complete melting, the alloy ingot was subjected to spray casting into a copper mold for rapid cooling and solidification within 4 min to obtain the alloy bar.

Hot working: The alloy bar was placed into a heating device, vacuumizing was performed, the alloy bar was heated to 950° C. at 10° C./min under rotation in an argon protective atmosphere, heat preservation was performed at the temperature for 30 min, and then the alloy bar was taken out for forging and rolling, where a deformation amount of the alloy bar was controlled at 10% during the process.

Heat treatment: The hot worked alloy bar was subjected to solid solution treatment and aging treatment, where the solid solution treatment included the following steps: cleaning a surface of the hot worked alloy bar to ensure that the surface was free of an impurity and an oxide layer, placing the cleaned alloy bar into a solid solution treatment device, heating up to 850° C. at a heating rate of 30° C. per hour in an argon protective atmosphere, and performing heat preservation at the temperature for 2.5 h; and the aging treatment included the following steps: taking the solid solution treated alloy bar out of the solid solution treatment device, performing surface cleaning, placing the cleaned alloy bar into an aging treatment device, heating up to 500° C. at a heating rate of 10° C. per hour in an argon protective atmosphere, performing heat preservation at the temperature for 3.5 h, and after the heat preservation was completed, naturally cooling the alloy bar to obtain the Ti70Zr20Nb5Cu2Ag1O2 alloy.

### Example 2

### Preparation of a Ti60Zr25Nb6Cu3Ag3O3 Alloy

Batching: 2828.73 g of a titanium elementary substance, 2303.54 g of a zirconium elementary substance, 563.09 g of a niobium elementary substance, 192.58 g of a copper elementary substance, 326.88 g of a silver elementary substance, and 121.02 g of titanium dioxide were weighed and uniformly mixed to obtain raw materials for melting, where purities of various elements by mass percentage were not lower than 99.0%.

Preparation of an alloy ingot using a vacuum arc furnace: The raw materials for melting were placed into the vacuum arc furnace, vacuumizing was performed to 7×10−3 Pa, argon was introduced, arc melting was performed in an argon protective atmosphere, and the alloy ingot was taken out and obtained, where a current was set at 85 A, a melting temperature was 2,000° C., a melting frequency was 5 times, and a melting time was 3 min each time.

Preparation of an alloy bar by solidification: The alloy ingot was placed into a rapid solidification device, vacuumizing was performed to 7×10-3 Pa, argon was introduced, the alloy ingot was heated to 2,000° C. in an argon protective atmosphere, and after complete melting, the alloy ingot was subjected to spray casting into a copper mold for rapid cooling and solidification within 2 min to obtain the alloy bar.

Hot working: The alloy bar was placed into a heating device, vacuumizing was performed, the alloy bar was heated to 1,000° C. at 15° C./min under rotation in an argon protective atmosphere, heat preservation was performed at the temperature for 45 min, and then the alloy bar was taken out for forging and rolling, where a deformation amount of the alloy bar was controlled at 20% during the process.

Heat treatment: The hot worked alloy bar was subjected to solid solution treatment and aging treatment, where the solid solution treatment included the following steps: cleaning a surface of the hot worked alloy bar to ensure that the surface was free of an impurity and an oxide layer, placing the cleaned alloy bar into a solid solution treatment device, heating up to 900° C. at a heating rate of 35° C. per hour in an argon protective atmosphere, and performing heat preservation at the temperature for 3 h; and the aging treatment included the following steps: taking the solid solution treated alloy bar out of the solid solution treatment device, performing surface cleaning, placing the cleaned alloy bar into an aging treatment device, heating up to 550° C. at a heating rate of 15° C. per hour in an argon protective atmosphere, performing heat preservation at the temperature for 4 h, and after the heat preservation was completed, naturally cooling the alloy bar to obtain the Ti60Zr25Nb6Cu3Ag3O3 alloy.

### Example 3

### Preparation of a Ti50Zr30Nb7Cu4Ag5O4 Alloy

Batching: 2321.03 g of a titanium elementary substance, 2764.24 g of a zirconium elementary substance, 656.94 g of a niobium elementary substance, 256.77 g of a copper elementary substance, 544.8 g of a silver elementary substance, and 161.35 g of titanium dioxide were weighed and uniformly mixed to obtain raw materials for melting, where purities of various elements by mass percentage were not lower than 99.0%.

Preparation of an alloy ingot using a vacuum arc furnace: The raw materials for melting were placed into the vacuum arc furnace, vacuumizing was performed to 9×10-3 Pa, argon was introduced, arc melting was performed in an argon protective atmosphere, and the alloy ingot was taken out and obtained, where a current was set at 120 A, a melting temperature was 2,500° C., a melting frequency was 5 times, and a melting time was 3 min each time.

Preparation of an alloy bar by solidification: The alloy ingot was placed into a rapid solidification device, vacuumizing was performed to 9×10-3 Pa, argon was introduced, the alloy ingot was heated to 2,500° C. in an argon protective atmosphere, and after complete melting, the alloy ingot was subjected to spray casting into a copper mold for rapid cooling and solidification within 3 min to obtain the alloy bar.

Hot working: The alloy bar was placed into a heating device, vacuumizing was performed, the alloy bar was heated to 1,050° C. at 20° C./min under rotation in an argon protective atmosphere, heat preservation was performed at the temperature for 60 min, and then the alloy bar was taken out for forging and rolling, where a deformation amount of the alloy bar was controlled at 30% during the process.

Heat treatment: The hot worked alloy bar was subjected to solid solution treatment and aging treatment, where the solid solution treatment included the following steps: cleaning a surface of the hot worked alloy bar to ensure that the surface was free of an impurity and an oxide layer, placing the cleaned alloy bar into a solid solution treatment device, heating up to 950° C. at a heating rate of 40° C. per hour in an argon protective atmosphere, and performing heat preservation at the temperature for 3.5 h; and the aging treatment included the following steps: taking the solid solution treated alloy bar out of the solid solution treatment device, performing surface cleaning, placing the cleaned alloy bar into an aging treatment device, heating up to 600° C. at a heating rate of 20° C. per hour in an argon protective atmosphere, performing heat preservation at the temperature for 4.5 h, and after the heat preservation was completed, naturally cooling the alloy bar to obtain the Ti50Zr30Nb7Cu4Ag5O4 alloy.

### Example 4

The difference between the present example and Example 2 is that a process step of coating a zinc-strontium three-dimensional network coating was added based on Example 2. That is, 25 parts of a 5 wt % calcium silicate solution, 5 parts of a 0.5 wt % zinc nitrate solution, and 5 parts of a 0.5 wt % strontium nitrate solution were uniformly mixed to form a uniform sol solution, 45 parts of ethanol was added to adjust viscosity and uniformity of the sol solution, the high-strength and low-modulus antibacterial alloy was immersed in the sol solution for 3 min and then slowly lifted to make the sol solution uniformly coated on a surface of the high-strength and low-modulus antibacterial alloy, natural air drying was performed, then the antibacterial alloy was placed into a heating device for heating to 600° C., and heat preservation was performed for 1 h to increase the zinc-strontium three-dimensional network coating on the surface of the Ti60Zr25Nb6Cu3Ag3O3 alloy.

### Example 5

The difference between the present example and Example 2 is that a process step of coating a zinc-strontium three-dimensional network coating was added based on Example 2. That is, 30 parts of a 17 wt % calcium silicate solution, 7 parts of a 3 wt % zinc nitrate solution, and 8 parts of a 3 wt % strontium nitrate solution were uniformly mixed to form a uniform sol solution, 55 parts of ethanol was added to adjust viscosity and uniformity of the sol solution, the high-strength and low-modulus antibacterial alloy was immersed in the sol solution for 4 min and then slowly lifted to make the sol solution uniformly coated on a surface of the high-strength and low-modulus antibacterial alloy, natural air drying was performed, then the antibacterial alloy was placed into a heating device for heating to 700° C., and heat preservation was performed for 2 h to increase the zinc-strontium three-dimensional network coating on the surface of the Ti60Zr25Nb6Cu3Ag3O3 alloy.

### Example 6

The difference between the present example and Example 2 is that a process step of coating a zinc-strontium three-dimensional network coating was added based on Example 2. That is, 35 parts of a 30 wt % calcium silicate solution, 9 parts of a 5.5 wt % zinc nitrate solution, and 11 parts of a 5.5 wt % strontium nitrate solution were uniformly mixed to form a uniform sol solution, 65 parts of ethanol was added to adjust viscosity and uniformity of the sol solution, the high-strength and low-modulus antibacterial alloy was immersed in the sol solution for 5 min and then slowly lifted to make the sol solution uniformly coated on a surface of the high-strength and low-modulus antibacterial alloy, natural air drying was performed, then the antibacterial alloy was placed into a heating device for heating to 800° C., and heat preservation was performed for 3 h to increase the zinc-strontium three-dimensional network coating on the surface of the Ti60Zr25Nb6Cu3Ag3O3 alloy.

### Comparative Example 1

The difference between the present comparative example and Example 2 is that the titanium dioxide was not added.

### Comparative Example 2

The differences between the present comparative example and Example 2 are that the titanium dioxide was not added, and all the preparation process steps were carried out in air without vacuumizing and argon introduction operations.

### Comparative Example 3

The differences between the present comparative example and Example 2 are that during the preparation of the alloy bar by solidification, after the complete melting, the spray casting into the copper mold for solidification was performed by adopting a natural cooling mode, and during the hot working, the deformation amount of the alloy bar was 35%.

### Test Methods

X-ray diffraction (XRD) analysis: An X-ray diffractometer with the model of D/MAX-2200pc, manufactured by Rigaku, Japan, was used with a Cu target (Kα radiation wavelength λ=1.5406 Å) as a target material. A sample was fixed to a center area of a sample holder by plasticine, and gently pressed by a glass slide to ensure that a sample surface and the sample holder were on a same horizontal plane. Parameters were set as follows: a working voltage was 40 KV, a working current was 40 mA, a scanning rate was 6°/min, and a scanning angle range was 20-100°.

Test method for mechanical properties of an alloy: A quasi-static compression experiment was carried out on a universal testing machine for materials with the model of SANS 5504 50KN. A rod-shaped alloy sample with a diameter of 2 mm was used, where an aspect ratio of the sample was about 2:1. Two ends of the sample were ground to be parallel to each other and perpendicular to an axis of the sample. During the experiment, a test speed was loaded at 2.1×10-4 s-1 through displacement control. Yield strength, fracture strength, and elastic modulus were obtained by calculation according to a resulting stress-strain curve.

Test method for corrosion resistance of an alloy: An electrochemical test was carried out in electrochemical workstations with the models of Princeton Applied Research Model VersaSTAT and VersaSTAT3. A three-electrode system was adopted, in which an alloy sample was used as a working electrode (WE), a platinum sheet was used as a counter electrode (CE), a saturated calomel electrode (SCE) was used as a reference electrode, and a solution was a PBS solution (phosphate buffer solution, NaCl-8.01, Kcl-0.20, Na2HPO4-1.15, KH2PO4-0.20, unit: g/L). The alloy sample with a diameter of 5 mm and a thickness of 2 mm was immersed in the solution until an open circuit potential (OCP) reached a stable state, and then a polarization experiment was carried out, including potentiodynamic polarization or cyclic anodic polarization. Potentiodynamic polarization scanning was started from the open circuit potential to 50 mV or below (OCP-50 mV), a stopping potential was set at 2.0 V, the experiment was manually stopped after pitting occurred, and a potential scanning rate was 50 m V/min.

Test method for an antibacterial effect of an alloy: 1 ml of a Staphylococcus aureus ATCC25923 (S. aureus) bacterial solution with a concentration of 1.5×106 cells/ml was added into a 24-well plate, and an alloy sample with a diameter of 2 mm and a length of 6 mm was placed into each well of the 24-well plate and completely immersed in the bacterial solution. The 24-well plate was placed into an incubator for culture at 37° C. for 24 h, and then the sample was taken out. The bacterial solution into which the sample was immersed in each culture well was diluted with a PBS solution (phosphate buffer solution, NaCl-8.01, KCl-0.20, Na2HPO4-1.15, KH2PO4-0.20, unit: g/L) at a concentration gradient of 1:100, 1:1000, and 1:10000. 100 μl of the diluted bacterial solution was sucked onto a nutrient agar plate and evenly spread on the plate, the plate was placed into an incubator for culture at 37° C. for 24 h, and discrete bacterial colonies visible to the naked eyes grew on a surface of the agar plate, where each single bacterial colony represented
one bacterium in the original sample. Numbers of bacterial colonies in culture dishes of different samples were recorded by a digital camera, and then compared with a negative control group to calculate antibacterial rates.

Performance tests were carried out on the alloys prepared in Examples 1-6 and Comparative Examples 1-3 according to the above test methods. Test results are shown in Table 1.

TABLE 1
Performance test results of alloys

| | Yield | Fracture | Elastic | Self-corrosion | Pitting | Antibacterial |
|---|---|---|---|---|---|---|
| | strength | strength | modulus | potential | potential | rate |
| Item | (MPa) | (MPa) | (GPa) | (mV) | (V) | (%) |
| Example 1 | 1086 | 1633 | 54 | 0.48 | 0.28 | 96.7 |
| Example 2 | 1150 | 1640 | 50 | 0.51 | 0.16 | 98.6 |
| Example 3 | 1064 | 1615 | 52 | 0.42 | 0.35 | 97.2 |
| Example 4 | 1155 | 1717 | 53 | 1.66 | 0.94 | 98.9 |
| Example 5 | 1167 | 1742 | 55 | 1.72 | 0.98 | 99.4 |
| Example 6 | 1195 | 1729 | 51 | 1.69 | 0.96 | 98.3 |
| Comparative | 650 | 758 | 98 | −0.63 | −3.68 | 75.8 |
| Example 1 | | | | | | |
| Comparative | 724 | 783 | 89 | −0.45 | −2.36 | 86.1 |
| Example 2 | | | | | | |
| Comparative | 688 | 769 | 81 | −0.57 | −4.52 | 97.9 |
| Example 3 | | | | | | |
| Ti—6Al—4V | 850 | 900 | 75 | −0.11 | 0.8 | 90.3 |

From the experimental data in Table 1, it can be seen that the high-strength and low-modulus antibacterial alloy of the present invention exhibits excellent performance in various properties.

All the alloys in Examples 1-6 have a yield strength of more than 1,000 MPa, a fracture strength of more than 1,500 MPa, an elastic modulus of lower than 55 GPa, and an antibacterial rate of higher than 96%, as well as a higher self-corrosion potential and a higher pitting potential, indicating that the alloys have high strength, low modulus, excellent antibacterial properties, and good corrosion resistance.

Through comparison between Examples 1-3, it can be seen that various properties are better in Example 2. This may be related to a composition ratio of various components in the alloy of Example 2 and setting of process parameters. In terms of composition of the components, atomic percentages of Ti, Zr, Nb, Cu, Ag, and O in Example 2 are 60, 25, 6, 3, 3, and 3, respectively. This ratio facilitates an interaction of various elements and promotes uniform distribution of a strengthening phase. In the process, during the preparation of the alloy ingot in the vacuum arc furnace, the current is 85 A, the melting temperature is 2,000° C., the melting frequency is 5 times, and the melting time is 3 min each time, so that the components of the alloy are fully melted, thereby reducing segregation of the components and ensuring homogeneity of the alloy. The rapid cooling and solidification within 2 min are conducive to forming a finer and more uniform grain structure, and refined grains can enhance strength and toughness of the alloy. During the hot working, the heating was performed to 1,000° C. at 15° C./min, the heat preservation was performed for 45 min, and the deformation amount was controlled at 20%, so that not only is the alloy ensured to have good plasticity to facilitate the forging and rolling, but also the grains can be effectively refined, thereby improving the strength and toughness of the alloy. Due to appropriate parameters of the solid solution treatment and the aging treatment, solute atoms are fully dissolved, and a precipitate phase is fully formed, thereby further optimizing a microstructure of the alloy and improving comprehensive performance of the alloy.

According to comparison between Examples 4-6 and Example 2, after the zinc-strontium three-dimensional network coating is added, the yield strength, the fracture strength, the self-corrosion potential, and the pitting potential are increased, and the antibacterial rate is increased. This is because the coating forms a stable three-dimensional network structure through ion exchange, which enhances a bonding force with the alloy surface, enables uniform distribution of zinc and strontium ions, and synergistically acts with copper and silver elements to improve antibacterial properties and corrosion resistance of the alloy.

Through continuous analysis of the experimental data, it can be found that compared with Example 2, the absence of titanium dioxide in Comparative Example 1 leads to decreases in the yield strength, the fracture strength, the self-corrosion potential, and the pitting potential, an increase in the elastic modulus, and an obvious decrease in the antibacterial rate. This is because the titanium dioxide, as a controllable oxygen source, reacts with other components at a high temperature to generate nano-oxides, and these nano-oxides serve as second-phase particles and are uniformly dispersed in an alloy matrix to play a grain refinement role. A fine grain strengthening mechanism increases obstacles to dislocation movement of the alloy, thereby significantly improving the yield strength and fracture strength of the alloy. Meanwhile, a fine grain structure increases a grain boundary area, which can effectively reduce the elastic modulus of the alloy and make the alloy closer to mechanical properties of natural bones of the human body, thereby reducing a stress shielding effect. In addition, the titanium dioxide has photocatalytic activity, which can generate hydroxyl free radicals to destroy bacterial structures. Meanwhile, oxygen vacancies generated by thermal decomposition of the titanium dioxide promote sustained release of silver and copper ions in the alloy, and a synergy of the two significantly improves antibacterial properties of the alloy.

In Comparative Example 2, although there is an oxygen source, the oxygen source is uncontrollable, leading to a decrease in the strength of the alloy, an increase in the modulus, and a decrease in the antibacterial rate, indicating that the combination of vacuum and argon protection with the controllable oxygen source is crucial for improving properties of the alloy. The vacuum and the argon can prevent alloy oxidation to ensure stability of the components and the microstructure and facilitate function utilization of the oxygen source.

In Comparative Example 3, the natural cooling is adopted during the solidification, and the deformation amount during the hot working reaches 35%, so that the yield strength, the fracture strength, the self-corrosion potential, and the pitting potential are increased, and the elastic modulus is increased. This may be because of the natural cooling, a cooling rate is low, and atoms have sufficient time to diffuse, so that grains undergo mutual annexation during growth and ultimately form coarse grains. The coarse grains decrease a grain boundary area and weaken a hindering effect of a grain boundary on dislocation movement, leading to decreases in the yield strength and fracture strength of the alloy. Meanwhile, a coarse grain structure is not conducive to dispersion of internal stress in the alloy, and is more prone to generating stress concentration under a load, thereby further reducing mechanical properties of the alloy. During the hot working, the excessive deformation amount exceeds a reasonable acceptable range of the alloy, which leads to a large number of defects and residual stress generated in the alloy, thereby reducing the strength and corrosion resistance of the alloy, destroying the internal microstructure of the alloy, and increasing the elastic modulus. Compared with a commonly used medical titanium alloy Ti-6A1-4V, the alloys in Examples 1-6 have superior yield strength, fracture strength, and antibacterial properties, as well as lower elastic modulus, and better meet requirements for mechanical properties of bones of the human body, indicating that the alloy of the present invention has significant performance advantages.

In summary, by adding the titanium dioxide, adopting the vacuum and argon protection preparation process, and coating the zinc-strontium three-dimensional network coating, the mechanical properties, corrosion resistance, and antibacterial properties of the alloy can be significantly improved.

The above only shows embodiments of the present invention and is not intended to limit the present invention. For those skilled in the art, various modifications and changes may be made to the present invention. Any modifications, equivalent substitutions, improvements, and the like made within the spirit and principles of the present invention shall be included within the scope of the claims of the present invention.

What is claimed is:

1. A high-strength and low-modulus antibacterial alloy, wherein the alloy has a molecular formula of TiₐZr_{b}Nb_{c}Cu_{d}AgₑO_{f}, wherein subscripts a, b, c, d, e, and f represent atomic percentages of corresponding alloy elements, with 50≤a≤70, 20≤b≤30, 5≤c≤7, 2≤d≤4, 1≤e≤5, 2≤f≤4, and a+b+c+d+e+f=100; and a remainder is an unavoidable impurity.

2. The high-strength and low-modulus antibacterial alloy according to claim 1, wherein a surface of the high-strength and low-modulus antibacterial alloy is coated with a zinc-strontium three-dimensional network coating.

3. A method for preparing the high-strength and low-modulus antibacterial alloy according to claim 1, comprising the following steps:
batching: weighing Ti, Zr, Nb, Ag, Cu, and TiO₂ according to preset target components of the alloy, and uniformly mixing the same to obtain raw materials for melting, wherein purities of various elements by mass percentage are not lower than 99.0%;
preparation of an alloy ingot using a vacuum arc: placing the raw materials for melting into a vacuum arc device, performing vacuumizing and arc melting in an argon protective atmosphere, and taking out and obtaining the alloy ingot;
preparation of an alloy bar by solidification: placing the alloy ingot into a rapid solidification device, performing vacuumizing and melting in an argon protective atmosphere, and after complete melting, performing spray casting into a copper mold for rapid cooling and solidification within 2-4 min to obtain the alloy bar;
hot working: placing the alloy bar into a heating device, performing vacuumizing, heating up to 950-1,050° C. at 10-20° C./min under rotation in an argon protective atmosphere to ensure uniform heating of the alloy bar, performing heat preservation at such a temperature for 30-60 min, and then taking out the alloy bar for forging and rolling, wherein a deformation amount of the alloy bar is controlled at 10-30% during the process; and
heat treatment: subjecting the hot worked alloy bar to solid solution treatment and aging treatment.

4. The method for preparing the high-strength and low-modulus antibacterial alloy according to claim 3, wherein during the preparation of the alloy ingot using the vacuum arc, a current is set at 50-120 A, a melting temperature is 1,500-2,500° C., a melting frequency is 4-5 times, and a melting time is 2-3 min each time.

5. The method for preparing the high-strength and low-modulus antibacterial alloy according to claim 3, wherein the solid solution treatment comprises the following steps: cleaning a surface of the alloy bar to ensure that the surface is free of an impurity and an oxide layer; placing the cleaned alloy bar into a solid solution treatment device, and heating up to 850-950° C. at a heating rate of 30-40° C. per hour in an argon protective atmosphere, wherein the heating rate is not too high to avoid thermal stress caused by a rapid temperature change; performing heat preservation at such a temperature for 2.5-3.5 h, wherein the heat preservation time is not too short to ensure that solute atoms inside the alloy are fully dissolved into a matrix to form a uniform single-phase solid solution; and after the heat preservation is completed, naturally cooling the alloy bar.

6. The method for preparing the high-strength and low-modulus antibacterial alloy according to claim 5, wherein the aging treatment comprises the following steps: taking the solid solution treated alloy bar out of the solid solution treatment device, and performing surface cleaning; placing the cleaned alloy bar into an aging treatment device, heating up to 500-600° C. at a heating rate of 10-20° C. per hour in an argon protective atmosphere, and performing heat preservation at such a temperature for 3.5-4.5 h, wherein the heat preservation time is not too short to ensure that a precipitate phase is formed inside the alloy to further improve material strength and hardness; and after the heat preservation is completed, naturally cooling the alloy bar.

7. A method for preparing the high-strength and low-modulus antibacterial alloy according to claim 2, comprising the following steps:
batching: weighing Ti, Zr, Nb, Ag, Cu, and TiO₂ according to preset target components of the alloy, and uniformly mixing the same to obtain raw materials for melting, wherein purities of various elements by mass percentage are not lower than 99.0%;
preparation of an alloy ingot using a vacuum arc: placing the raw materials for melting into a vacuum arc device, performing vacuumizing and arc melting in an argon protective atmosphere, and taking out and obtaining the alloy ingot;
preparation of an alloy bar by solidification: placing the alloy ingot into a rapid solidification device, performing vacuumizing and melting in an argon protective atmosphere, and after complete melting, performing spray casting into a copper mold for rapid cooling and solidification within 2-4 min to obtain the alloy bar;
hot working: placing the alloy bar into a heating device, performing vacuumizing, heating up to 950-1,050° C. at 10-20° C./min under rotation in an argon protective atmosphere to ensure uniform heating of the alloy bar, performing heat preservation at such a temperature for 30-60 min, and then taking out the alloy bar for forging and rolling, wherein a deformation amount of the alloy bar is controlled at 10-30% during the process;
heat treatment: subjecting the hot worked alloy bar to solid solution treatment and aging treatment; and
coating with a zinc-strontium three-dimensional network coating: uniformly mixing, in parts by mass, 25-35 parts of a 5-30 wt % calcium silicate solution, 5-9 parts of a 0.5-5.5 wt % zinc nitrate solution, and 5-11 parts of a 0.5-5.5 wt % strontium nitrate solution to form a uniform sol solution, adding 45-65 parts of ethanol to adjust viscosity and uniformity of the sol solution, immersing the high-strength and low-modulus antibacterial alloy in the sol solution for 3-5 min, then slowly lifting the antibacterial alloy to make the sol solution uniformly coated on a surface of the high-strength and low-modulus antibacterial alloy, performing natural air drying, then placing the antibacterial alloy in a heating device for heating to 600-800° C., and performing heat preservation for 1-3 h.

8. Use of the high-strength and low-modulus antibacterial alloy according to claim 1 in an orthopedic or dental material.

9. Use of the high-strength and low-modulus antibacterial alloy according to claim 2 in an orthopedic or dental material.

10. Use of the high-strength and low-modulus antibacterial alloy obtained by the preparation method according to claim 7 in an orthopedic or dental material.
